(19) United States
(12) Patent Application Publication
(10) Pub. No.: US 2020/0197012 A1
(43) Pub. Date: Jun. 25, 2020

(54) CLOSED END-TYPE CEREBRAL ANEURYSM CLIP AND MANUFACTURING METHOD THEREFOR

(71) Applicant: Hyo Joon KIM, Jeollabuk-do (KR)

(72) Inventors: Hyo Joon KIM, Jeollabuk-do (KR); Yoon Won LEE, Seoul (KR)

(21) Appl. No.: 16/616,639

(22) PCT Filed: May 29, 2017
(86) PCT No.: PCT/KR2017/005575

§ 371 Date: Nov. 25, 2019

(51) Int. Cl. A61B 17/122; A61B 17/128

(52) U.S. Cl. CPC A61B 17/122; A61B 17/128

(57) ABSTRACT

A cerebral aneurysm clip for ligating a cerebral aneurysm includes a first ligation part having a first hole and having a closed end shape in which an edge of the first hole is closed; a second ligation part having a second hole and having a closed end shape in which an edge of the second hole is closed; and a tong part connected with the first and second ligation parts and applying a force such that they may be ligated on the cerebral aneurysm. The first and second ligation parts are arranged to face each other, such that the first and second ligation parts are ligated on both sides, respectively, with the cerebral aneurysm therebetween, the first and second holes communicate with each other, and the cerebral aneurysm is positioned and ligated in the first and second holes.

### TECHNICAL FIELD

The present invention relates to a clip used to ligate a cerebral aneurysm and a method of manufacturing the clip. More particularly, the present invention relates to a closed end-type cerebral aneurysm clip and a manufacturing method thereof, in which a ligation part of the cerebral aneurysm clip is formed in the shape of a closed end such as a loop shape such that ends of the ligation part face each other to perform the function of tongs, thus enhancing safety and convenience when a blood vessel or a cerebral aneurysm is ligated, and increasing a ligation force to improve a success rate for ligation.

### BACKGROUND ART

An aneurysm is a disease in which a defect occurs in a part of an artery and the part swells like a balloon, thus forming an alveolar shape and then spontaneously bursting, and consequently causing death or serious damage. Conventionally, a blood vessel stent or a surgical method is used to block blood stream to the aneurysm or to block a bleeding region. Examples of the conventional surgical method which is widely used to block the blood stream to the aneurysm or to block the bleeding region include aneurysm ligation and aneurysm embolization. The aneurysm ligation used to treat the cerebral aneurysm is a method of incising the head, checking the location of the aneurysm through a microscope, accessing the aneurysm, and then ligating the neck portion of the aneurysm with very small tongs such as a clip.

The aneurysm ligation is difficult in the surgical procedure or process. However, even when the aneurysm is located too deep in the brain or is surrounded by a dangerous structure, the surgery is needed, so that higher safety is required. Particularly, when ligating the cerebral aneurysm, the balloon or the alveolar-shaped portion should be exposed and the neck portion should be ligated so as to reliably block the blood stream.

FIG. 1 is a diagram illustrating a state of ligating a cerebral aneurysm 3 using a conventional cerebral aneurysm clip 1. The cerebral aneurysm 3 swells in the balloon or alveolar shape from a blood vessel 4 unlike the general blood vessel 4.

Conventionally, a ligation part of the cerebral aneurysm clip 1, namely, a blade tip 2 of the cerebral aneurysm clip 1 is configured in an open end shape, that is, in a shape that is opened like tweezers or tongs. This may increase the risk or make a surgical process complicated when the blood vessel or the cerebral aneurysm 3 is ligated. For example, especially when adhesion is severe between the blood vessel or the cerebral aneurysm and the peripheral tissue and thus dissection itself is dangerous, the conventional cerebral aneurysm clip 1 with the open end-type blade tip 2 may make the ligation incomplete or impossible. Further, since the blade tip 2 is sharp, it may undesirably cause damage to the blood vessel.

Further, conventionally, when ligating the cerebral aneurysm 2 that swells like the balloon or is formed in the alveolar shape, an additional dissection process for removing surrounding impurities should be performed to precisely ligate the neck of the cerebral aneurysm, so that an operator should accept risk due to the dissection process. Moreover, since the conventional cerebral aneurysm clip 1 with the open end-type blade tip 2 may clip or ligate other adjacent blood vessels 4 as well as the cerebral aneurysm 3. In order to solve the problem, a dangerous process should be performed: the cerebral aneurysm is pulled to be checked with a mirror or a contrast medium is injected.

Furthermore, due to the structure of the conventional cerebral aneurysm clip with the open end-type blade tip 2, it is difficult to treat a giant aneurysm which is difficult to find a dissection portion, an aneurysm origin or the like and a giant aneurysm from which the blood vessel that is the cerebral-aneurysm origin is invisible, and the ligation for hard cerebral aneurysm such as arteriosclerosis may be loosened, so that stability cannot be ensured.

### DISCLOSURE

### Technical Problem

The present invention has been made to solve the above-mentioned problems and an object of the invention is to provide a closed end-type cerebral aneurysm clip, in which a ligation part of the cerebral aneurysm clip used as a ligation tool in a surgical process for a cerebral aneurysm through craniotomy or subarachnoidal hemorrhage is formed in the shape of a closed end such as a loop shape such that ends of the ligation part face each other to perform the function of tongs, thus enhancing safety and convenience when a blood vessel or a cerebral aneurysm is ligated, and increasing a ligation force to improve a success rate for ligation.

The invention is to provide a closed end-type cerebral aneurysm clip, in which a dissection process of a cerebral aneurysm swollen like a balloon or formed in an alveolar shape is omitted or remarkably reduced when the cerebral aneurysm is ligated, and then a neck of the cerebral aneurysm may be precisely ligated, thus allowing a surgical process to be more safely performed.

The invention is to provide a closed end-type cerebral aneurysm clip, which prevents a tip of the cerebral aneurysm clip for ligating a cerebral aneurysm in a process for ligating the cerebral aneurysm, namely, a ligation part of the cerebral aneurysm clip from clipping or ligating blood vessels other than the cerebral aneurysm that is to be ligated.

The invention is to provide a closed end-type cerebral aneurysm clip, which makes it easy to treat a giant aneurysm which is difficult to find a dissection portion, an aneurysm origin or the like and a giant aneurysm from which the blood vessel that is the cerebral-aneurysm origin is invisible.

Particularly, the invention is to provide a cerebral aneurysm clip, which omits or simplifies a dissection work for removing impurities around a cerebral aneurysm, when the cerebral aneurysm is formed in a point where blood vessels are branched in a ‘y’ shape, thus making it convenient to ligate the cerebral aneurysm.

The invention is to provide a closed end-type cerebral aneurysm clip, in which a ligation part of the cerebral aneurysm clip is formed in the shape of a closed end to apply a force to the ligation part and evenly maintain the force, thus preventing the ligation for hard cerebral aneurysm such as arteriosclerosis from being loosened and further enhancing stability.

### Technical Solution

The present invention provides a cerebral aneurysm clip.

According to the invention, the cerebral aneurysm clip for ligating a cerebral aneurysm includes: a first ligation part having a first hole formed therein, and having a closed end shape in which an edge of the first hole is closed; a second ligation part having a second hole formed therein, and having a closed end shape in which an edge of the second hole is closed; and a tong part connected with the first ligation part and the second ligation part, and applying a force such that the first ligation part and the second ligation part may be ligated on the cerebral aneurysm, wherein the first ligation part and the second ligation part are arranged to face each other, such that the first ligation part and the second ligation part are ligated on both sides, respectively, with the cerebral aneurysm therebetween, and the first hole and the second hole communicate with each other, and the cerebral aneurysm is positioned and ligated in the first hole and the second hole.

The tong part may have predetermined elasticity, and a distance between the first ligation part and the second ligation part may be increased if a force is applied to an external portion of the tong part, and the distance between the first ligation part and the second ligation part may be reduced if the applied force is removed from the external portion of the tong part.

The cerebral aneurysm clip may further include a first connection part connecting the first ligation part with the tong part; and

A second connection part connecting the second ligation part with the tong part, wherein the first connection part and the second connection part may intersect with each other.

The cerebral aneurysm clip may further include an elastic member coupled at both ends thereof to the first connection part and the second connection part, wherein the elastic member may provide an elastic force in a direction for reducing the distance between the first ligation part and the second ligation part, when the cerebral aneurysm is ligated on the first ligation part and the second ligation part.

The present invention provides a method of manufacturing a cerebral aneurysm clip.

According to the invention, the method of manufacturing the clip for ligating the cerebral aneurysm includes forming a first ligation part and a second ligation part, each having a closed end shape, on both sides of a base part having predetermined elasticity to be symmetrical with respect to each other; folding the base part such that the first ligation part and the second ligation part face each other; forming a tong part between the first ligation part and the second ligation part, and forming a first connection part connecting the first ligation part with the tong part, and a second connection part connecting the second ligation part with the tong part; and intersecting the first connection part and the second connection part with each other such that a front surface of the first ligation part and a rear surface of the second ligation part face each other.

The base part may be a straight line-shaped member having predetermined elasticity.

### Advantageous Effects

As described above, the closed end-type cerebral aneurysm clip of the present invention is configured such that the ligation part of the cerebral aneurysm clip used as the ligation tool in the surgical process for the cerebral aneurysm through the craniotomy or the subarachnoidal hemorrhage is formed in the shape of the closed end such as the loop shape such that the ends of the ligation part face each other to perform the function of the tongs, thus achieving the following effects.

By ligating the cerebral aneurysm with the closed end-type ligation structure in the surgical process for the cerebral aneurysm through the craniotomy, safety and convenience are enhanced when the cerebral aneurysm is ligated, and the ligation force is increased to improve the success rate for ligation.

The invention prevents a blood vessel from being damaged by the blade tip of the clip for ligating the cerebral aneurysm, thus allowing the cerebral aneurysm to be conveniently and safely ligated.

Particularly, the invention omits or simplifies the dissection work for removing the impurities adhered around the cerebral aneurysm, when the cerebral aneurysm is formed in a point where blood vessels are branched in a ‘y’ shape, thus making it convenient and safe to ligate the cerebral aneurysm.

Further, after the cerebral aneurysm is ligated, the clip is prevented from being opened again, so that the ligation state can be stably maintained.

### DESCRIPTION OF DRAWINGS

FIG. 1 is a diagram illustrating a state of ligating a cerebral aneurysm using a conventional cerebral aneurysm clip;

FIG. 2 is a perspective view showing a closed end-type cerebral aneurysm clip according to an embodiment of the present invention;

FIG. 3 is a perspective view showing a configuration wherein the closed end-type cerebral aneurysm clip shown in FIG. 2 is held and opened by tongs;

FIG. 4 is a perspective view showing an example wherein the closed end-type cerebral aneurysm clip shown in FIG. 3 is used in the form of tongs;

FIG. 5 is a perspective view showing another shape of the closed end-type cerebral aneurysm clip shown in FIG. 2;

FIG. 6 is a perspective view showing another shape of the closed end-type cerebral aneurysm clip shown in FIG. 2;

FIG. 7 is a perspective view showing another shape of the closed end-type cerebral aneurysm clip shown in FIG. 2;

FIG. 8 is a perspective view showing another shape of the closed end-type cerebral aneurysm clip shown in FIG. 2;

FIG. 9 is a perspective view showing another shape of the closed end-type cerebral aneurysm clip shown in FIG. 2;

FIG. 10 is a perspective view showing a further shape of the closed end-type cerebral aneurysm clip shown in FIG. 2;

FIG. 11 is a perspective view showing an example wherein the semi-circular closed end-type cerebral aneurysm clip shown in FIG. 8 is used in the form of tongs;

FIG. 12 is a perspective view showing an example wherein the diamond-shaped semi-circular closed end-type cerebral aneurysm clip is used in the form of tongs;

FIGS. 13 to 17 are diagrams illustrating various modifications of a tong part;

FIG. 18 is a flowchart illustrating a process of manufacturing a cerebral aneurysm clip according to the present invention;

FIG. 19 shows a method of manufacturing a cerebral aneurysm clip according to a first embodiment of the present invention; and

FIG. 20 shows a method of manufacturing a cerebral aneurysm clip according to a second embodiment of the present invention.

### BEST MODE

Hereinafter, embodiments of the present invention will be described in detail with reference to the accompanying drawings.

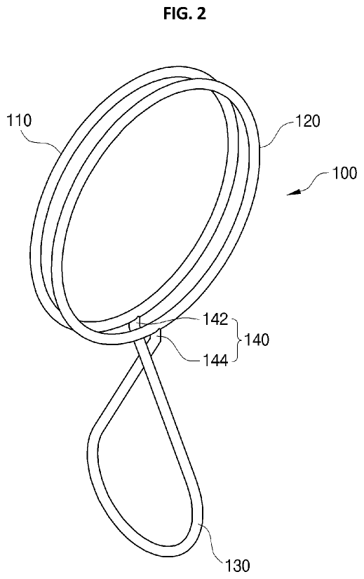

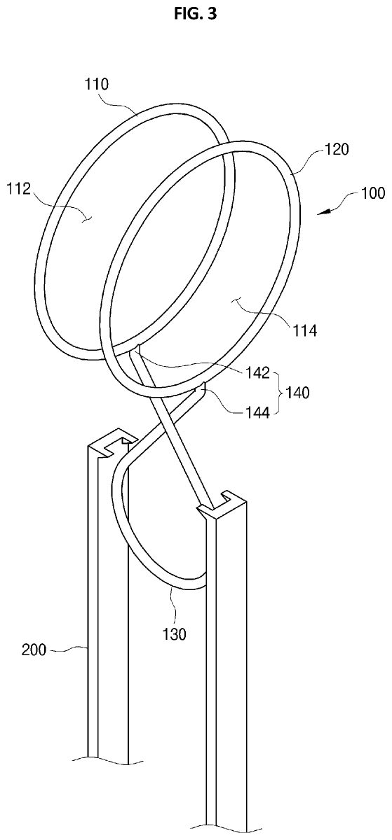

FIG. 2 is a perspective view showing a closed end-type cerebral aneurysm clip according to an embodiment of the present invention. FIG. 3 is a perspective view showing a configuration wherein the closed end-type cerebral aneurysm clip shown in FIG. 2 is opened.

Referring to FIGS. 2 and 3, the closed end-type cerebral aneurysm clip 100 includes a first ligation part 110, a second ligation part 120, a tong part 130, and a connection part 140.

The first ligation part 110 and the second ligation part 120 each retain a closed end shape including a loop shape to face each other.

A first hole 112 is formed in the first ligation part 110. An edge of the first hole 112 has the closed end shape that is closed along its circumference.

A second hole 122 is formed in the second ligation part 120. An edge of the second hole 122 has the closed end shape that is closed along its circumference.

Further, the edges of the first hole 112 and the second hole 122, namely, the edges of the first ligation part 110 and the second ligation part 120 may be formed to be round.

For example, each of the first ligation part 110 and the second ligation part 120 is configured to retain the closed end shape including an elliptical loop shape. The first ligation part 110 and the second ligation part 120 are connected by the tong part 130 to face each other. The first ligation part 110 and the second ligation part 120 are ligated on both sides of the cerebral aneurysm with the cerebral aneurysm therebetween.

The first hole 112 and the second hole 122 communicate with each other. The cerebral aneurysm that is to be ligated may be located in a communicating area. By way of example, if the neck of the cerebral aneurysm is ligated, the cerebral aneurysm may be visibly checked through the first hole 112 and the second hole 122 by naked eye.

The closed end-type first ligation part 110 and second ligation part 120 have the function of tongs via the tong part 130. The blood vessel such as the cerebral aneurysm is ligated between the first ligation part 110 and the second ligation part 120.

By way of example, the neck portion of the cerebral aneurysm may be ligated. Alternatively, the entire cerebral aneurysm as well as the neck portion of the cerebral aneurysm may be ligated.

When each of the first ligation part 110 and the second ligation part 120 retains the closed end shape, any one portion of each of the first ligation part 110 and the second ligation part 120 has no sharp portion, so that there is a low risk that the cerebral aneurysm will be ruptured during surgery. In addition, tips of the first ligation part 110 and the second ligation part 120 can be always seen, thus preventing a perforator artery from being clipped and blocked by mistake.

Particularly, the first ligation part 110 and the second ligation part 120 each retain a preset closed end shape such as a circular shape, an elliptical shape, a semi-circular shape, an inverted semi-circular shape, or a diamond shape, and are disposed such that fronts of the first and second ligation parts face each other. Thus, even when incomplete arterial dissection occurs, the ligation parts can fully ligate a blood vessel in conformity with its shape. Moreover, a surgery may be performed while repeating a partial ligating process without the risk of rupture even in the giant cerebral aneurysm from which the blood vessel that is the origin of the cerebral aneurysm is invisible.

The tong part 130 may be made in the shape of the loop that is connected with the first ligation part 110 and the second ligation part 120 that face each other. The tong part 130 provides the function of tongs and applies a force to retain the function of the tongs, thus allowing the first ligation part 110 and the second ligation part 120 to be ligated on the cerebral aneurysm. The tong part 130 may have predetermined elasticity. The tong part 130 may be configured in the shape of the loop wound in the shape of a coil at least once. When the tong part 130 has such a shape, the ligation force of the first ligation part 110 and the second ligation part 120 can be further enhanced.

The connection part 140 connects the first ligation part 110, the second ligation part 120, and the tong part 130. The connection part may include a first connection part 142 and a second connection part 144. The first connection part 142 connects the first ligation part 110 with the tong part 130. The second connection part 144 connects the second ligation part 120 with the tong part 130.

The first connection part 142 and the second connection part 144 may be configured to intersect with each other. Hence, this provides the function of tongs to the first ligation part 110 and the second ligation part 120, and maintains a force.

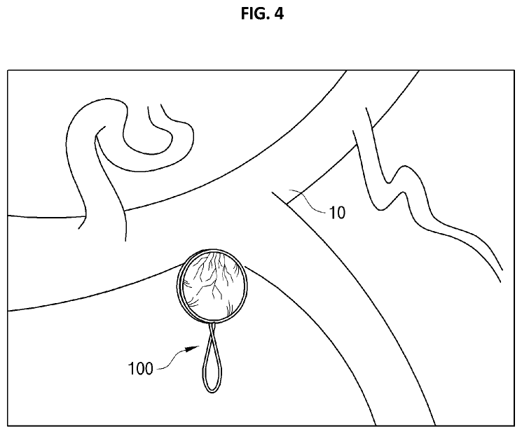

FIG. 4 is a perspective view showing an example wherein the closed end-type cerebral aneurysm clip shown in FIG. 3 is used in the form of tongs.

The first and second connection parts of the connection part 140 connected with the first ligation part 110 and the second ligation part 120 intersect with each other to form the tong part 130. In this state, if a force is applied to the external portion of the tong part 130 via tweezers or tongs 200, a distance between the first ligation part 110 and the second ligation part 120 is increased. In this state, at least some areas of the blood vessel including the cerebral aneurysm, for example, the neck portion of the cerebral aneurysm is positioned between the first ligation part 110 and the second ligation part 120. Thereafter, if the force applied to the external portion of the tong part 130 is eliminated, the distance between the first ligation part 110 and the second ligation part 120 is reduced, thus ligating the neck of the cerebral aneurysm. Alternatively, after the neck portion, the entire cerebral aneurysm, or any area of the cerebral aneurysm is located between the first ligation part 110 and the second ligation part 120, even if the force is removed from the tong part 130, the cerebral aneurysm may be easily ligated due to the large areas of the first ligation part 110 and the second ligation part 120.

If the first and second connection parts of the connection part 140 connected with the first ligation part 110 and the second ligation part 120 are configured to intersect with each other, the distance may be provided between the first ligation part 110 and the second ligation part 120 only by the force for pressing the tong part 130, and then the first ligation part 110 and the second ligation part 120 may perform the function of the tongs.

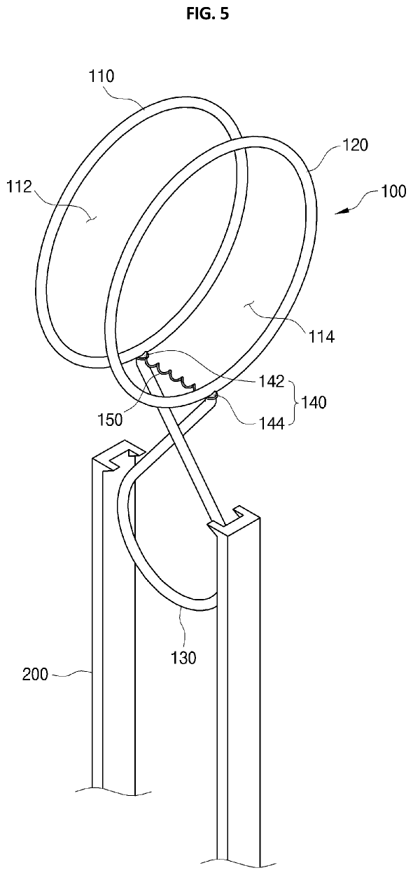

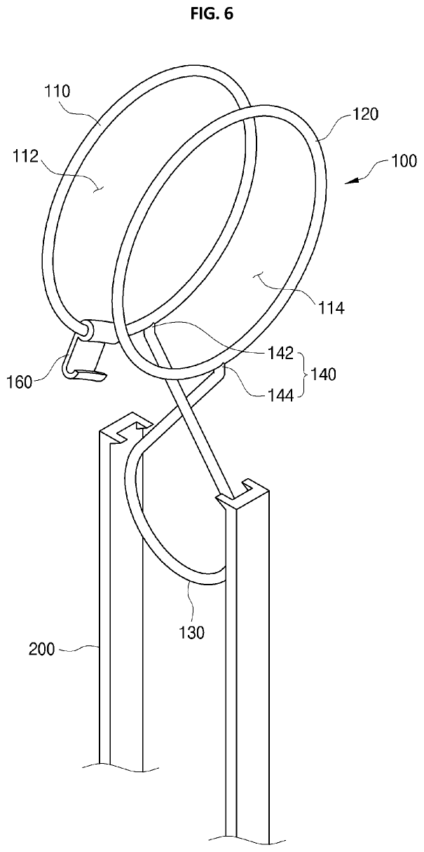

FIGS. 5 and 6 are diagrams showing another embodiment that is modified to reinforce the ligation force of the first ligation part and the second ligation part.

As shown in FIG. 5, an elastic member 150 may be provided between the first connection part 142 and the second connection part 144. The elastic member is fixedly coupled at both ends thereof to the first connection part 142 and the second connection part 144, so that an elastic force is provided between the first connection part and the second connection part.

After the cerebral aneurysm is ligated between the first ligation part 142 and the second ligation part 144, the distance between the first ligation part and the second ligation part is increased, so that the ligation force may be reduced. In this case, the elastic member 150 provides an elastic force in a direction where it contracts, so that the ligation force of the first ligation part 142 and the second ligation part 144 may be increased.

Alternatively, as shown in FIG. 6, a subsidiary fastening part 160 may be installed in either of the first ligation part 142 and the second ligation part 144. One side of the subsidiary fastening part 160 may be hingedly coupled to the first ligation part 110.

By way of example, one side of the subsidiary fastening part 160 may be hingedly coupled to an area adjacent to the first connection part 142 in the first ligation part 110. To be more specific, one side of the subsidiary fastening part 160 may be coupled to a side portion or a lower portion of the first ligation part 110. Generally, since an upper portion of the first ligation part 110 is used to ligate the neck of the cerebral aneurysm, this prevents the subsidiary fastening part 160 from obstructing the ligation.

If the cerebral aneurysm is ligated between the first ligation part 142 and the second ligation part 144, the subsidiary fastening part 160 is hingedly rotated, so that the other side of the subsidiary fastening part 160 may be fastened to the second ligation part 120. Therefore, since the first ligation part 110 and the second ligation part 120 are fastened to each other while the first ligation part 110 and the second ligation part 120 maintain a predetermined distance by the subsidiary fastening part 160, this may prevent the distance between the first ligation part 110 and the second ligation part 120 from increasing and reinforcing the ligation force.

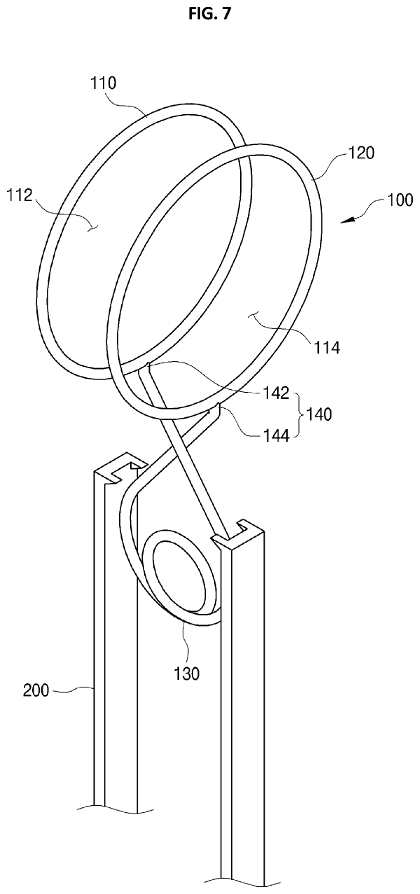

FIG. 7 is a perspective view showing another shape of the closed end-type cerebral aneurysm clip shown in FIG. 2.

The first ligation part 110 and the second ligation part 120 each retain a preset closed end shape such as a circular shape, an elliptical shape, a semi-circular shape, an inverted semi-circular shape, or a diamond shape. Here, the first ligation part 110 and the second ligation part 120 may be disposed such that fronts thereof face each other by the tong part 130. The tong part 130 is configured by intersecting the first and second connection parts of the connection part 140 that are connected with the first ligation part 110 and the second ligation part 120, and may be configured in the shape of the loop wound in the shape of a coil at least once. If the tong part 130 maintains the shape of the loop wound in the shape of the coil at least once, the ligation forces of the first ligation part 110 and the second ligation part 120 can be further increased.

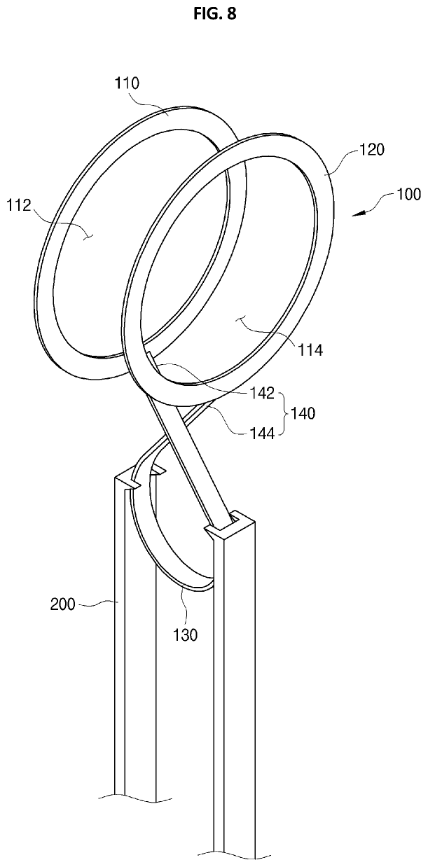

FIG. 8 is a perspective view showing another shape of the closed end-type cerebral aneurysm clip shown in FIG. 2.

As shown in FIG. 8, at least one of the first ligation part 110, the second ligation part 120, and the tong part 130 may be made in the shape of a plate having a preset area and length. For example, if the first ligation part 110 and the second ligation part 120 are made in the shape of the plate, the manufacturing method or process can be facilitated. If the tong part 130 is made in the shape of the plate, the ligation forces of the first ligation part 110 and the second ligation part 120 can be further increased.

Meanwhile, at least some areas of the blood vessel including the cerebral aneurysm requiring the ligation may be wide or narrow at the neck, or may be of various shapes that are not specified. Thus, the area where the first ligation part 110 and the second ligation part 120 face each other and then are ligated should be variously changed depending on the area or the shape of the corresponding neck. Thus, the first ligation part 110 and the second ligation part 120 are previously configured to retain at least one preset closed end shape among a circular shape, an elliptical shape, a semi-circular shape, an inverted semi-circular shape, a diamond shape, and a polygonal shape, so that the shape is selectively used regardless of the size and shape of the cerebral aneurysm requiring the ligation, an angle relative to another blood vessel, a positional relationship such as a relative position, etc.

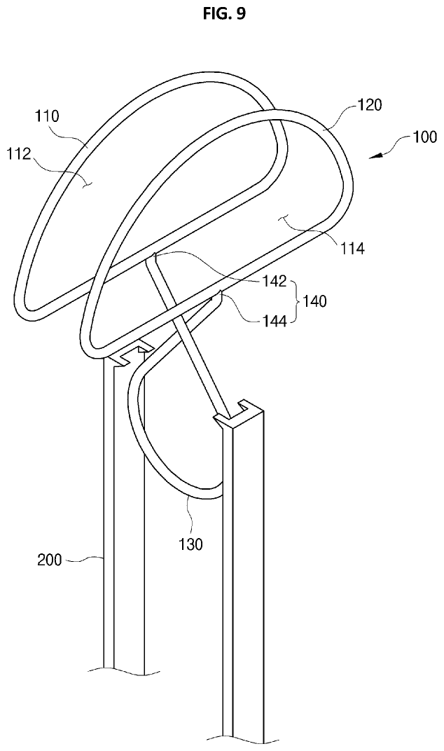

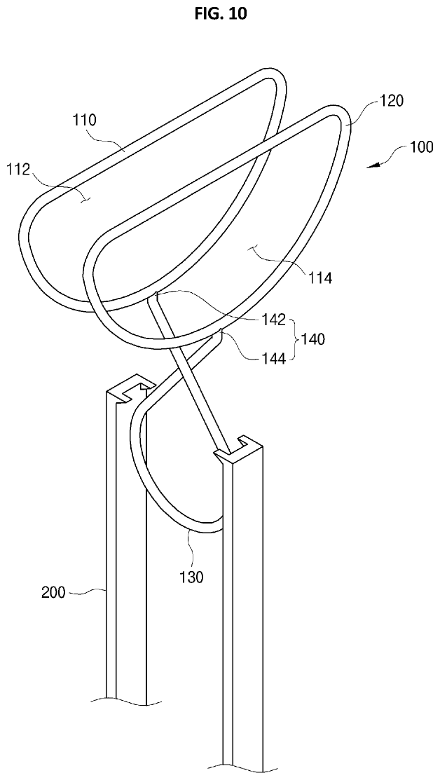

FIG. 9 is a perspective view showing another shape of the closed end-type cerebral aneurysm clip shown in FIG. 2, and FIG. 10 are perspective views showing a further shape of the closed end-type cerebral aneurysm clip shown in FIG. 2.

As shown in FIGS. 9 and 10, the first ligation part 110 and the second ligation part 120 each are configured in various shapes to retain a preset closed end shape such as a circular shape, a semi-circular shape, an inverted semi-circular shape, a crescent shape, or an inverted crescent shape, so that the shape may be selectively used regardless of the size and shape of the cerebral aneurysm requiring the ligation, an angle relative to another blood vessel, a positional relationship such as a relative position, etc.

At least some areas of the blood vessel including the cerebral aneurysm requiring the ligation may be wide or narrow at the neck, or may be of various shapes that are not specified. Thus, the first ligation part 110 and the second ligation part 120 are previously configured in various shapes to retain a preset closed end shape such as a circular shape, an elliptical shape, a semi-circular shape, an inverted semi-circular shape, a diamond shape, and a polygonal shape, so that the shape is selectively used regardless of the size and shape of the cerebral aneurysm requiring the ligation. It is difficult to ligate the cerebral aneurysm having a wide neck with the conventional tong- or straight-type clip that is generally utilized. However, the closed end-type cerebral aneurysm clip 100 may completely ligate the entire cerebral aneurysm including a wide neck.

Particularly, the area that is ligated by the first ligation part 110 and the second ligation part 120 facing each other may be variously changed according to the area or shape of the associated neck. If the ligation area facing the first ligation part 110 and the second ligation part 120 is variously changed, various shapes of cerebral aneurysms including a wide neck may be completely ligated.

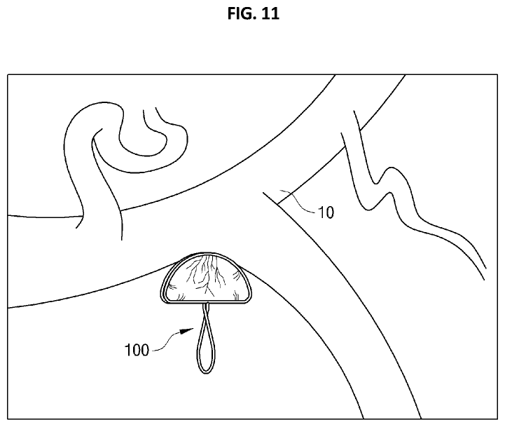

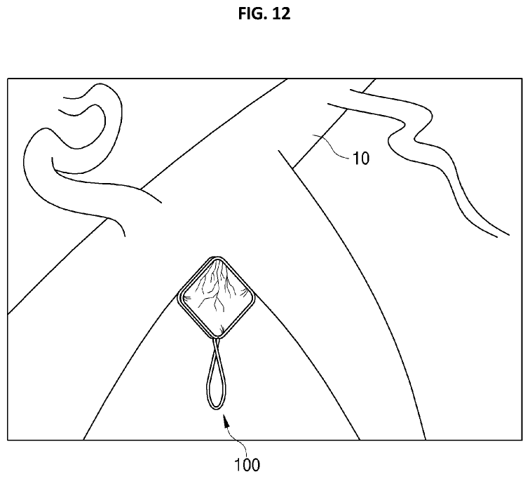

FIG. 11 is a perspective view showing an example wherein the semi-circular closed end-type cerebral aneurysm clip shown in FIG. 10 is used in the form of tongs. FIG. 12 is a perspective view showing an example wherein the diamond-shaped semi-circular closed end-type cerebral aneurysm clip is used in the form of tongs.

Referring to FIGS. 11 and 12, when there is severe adhesion between the cerebral aneurysm and the peripheral blood vessel 10, this may lead to tearing or rupture of the cerebral aneurysm during a dissection process, require a long time, and cause damage to peripheral brain and blood vessels. However, in the case of using the closed end-type cerebral aneurysm clip 100 as shown in FIGS. 11 and 12, safe and complete ligation of the cerebral aneurysm is possible without the dissection of an adhesion site. Particularly, after the dissection process of the cerebral aneurysm that is swollen like a balloon or is formed in an alveolar shape is omitted or remarkably reduced, the neck of the cerebral aneurysm can be precisely ligated.

In the state where the first ligation part 110 and the second ligation part 120 each retaining a specific shape such as a semi-circular shape or a diamond shape intersect with each other to form the tong part 130, if a force is applied to the external portion of the tong part 130 via the tweezers or tongs 200, the distance between the first ligation part 110 and the second ligation part 120 is increased. In this state, at least some areas of the blood vessel including the cerebral aneurysm are positioned between the first ligation part 110 and the second ligation part 120. Thereafter, if the force is not applied to the external portion of the tong part 130, the first ligation part 110 and the second ligation part 120 ligate the neck of the cerebral aneurysm.

Therefore, if the first ligation part 110 and the second ligation part have various shapes as shown in FIGS. 8 to 10, the cerebral aneurysm may be easily ligated even when the blood vessel is formed as shown in FIGS. 11 and 12.

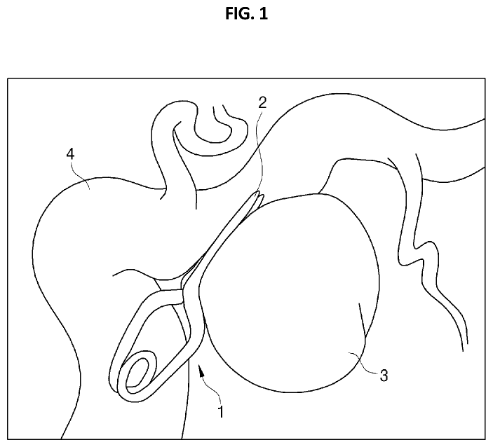

Particularly, as shown in FIGS. 4, 11, and 12, when the cerebral aneurysm is formed at a point where the blood vessels 10 branch from each other, it is very difficult to ligate the cerebral aneurysm with the conventional clip shown in FIG. 1. Unlike the general blood vessel, the cerebral aneurysm is formed to be swollen from the blood vessel in the shape of the balloon or alveolar, thus making it difficult to realize the safe and easy ligation by the ligation method for the general blood vessel.

Further, as shown in FIG. 1, in order to ligate the neck of the cerebral aneurysm with the conventional cerebral aneurysm clip, the blood vessel 10 should be approached from a side thereof. However, during this process, the blood vessel 10 may be damaged due to the sharp blade tip, and it is difficult to capture a point for ligation. Consequently, it is difficult to ligate the neck of the cerebral aneurysm.

In contrast, the cerebral aneurysm clip according to the present invention allows the cerebral aneurysm to be approached from all directions as well as the side of the blood vessel, so that it is easy to capture the point for ligation. Further, this clip is advantageous in that it is formed in the closed end type and is not sharp, so that there is no possibility of damaging other blood vessels.

Particularly, this clip may be formed in the closed end type while the edge of each of the ligation parts 110 and 120 is rounded. Such a structure is much more effective in preventing damage to other blood vessels.

Further, in some cases, the neck of the cerebral aneurysm may be excessively short or small, and may not be clearly visible. Thus, it is very difficult to ligate the cerebral aneurysm with the conventional cerebral aneurysm clip. However, even in this case, the cerebral aneurysm clip 100 according to the present invention is advantageous in that it is easy to capture the point for ligation and the easy ligation is possible.

Further, as shown in FIGS. 4, 11, and 12, when the blood vessels are branched in a ‘y’ shape or the like and the cerebral aneurysm is formed in a branch point, there may occur severe adhesion between the blood vessels and the cerebral aneurysm, so that various impurities including fat may be present as well. Therefore, in order to use the conventional clip shown in FIG. 1, a dissection work for removing these impurities is essential before the ligation is performed, so that the ligation process is very complicated and the blood vessel may be damaged. Further, although the dissection work has been performed, it is difficult to precisely ligate the neck of the cerebral aneurysm with the open end-type cerebral aneurysm clip in the shape of the blade tip.

However, the cerebral aneurysm clip according to the present invention is an innovative invention that allows the cerebral aneurysm to be conveniently, safely, and immediately ligated, without requiring the dissection work for removing these impurities. Further, if the closed type cerebral aneurysm clip is manufactured to have the shape corresponding to that of the blood vessels branched in the y shape, the cerebral aneurysm can be conveniently and safely ligated without requiring the complicated dissection process or ligation process.

As such, the closed end-type cerebral aneurysm clip according to the present invention is configured to ligate the cerebral aneurysm with the closed end-type ligation structure in the cerebral aneurysm surgery through craniotomy, thus enhancing safety and convenience in the cerebral-aneurysm ligation process, and increasing a ligation force, and consequently increasing a success rate for ligation. Moreover, in the case of ligating the cerebral aneurysm, after the dissection process of the cerebral aneurysm that is swollen like the balloon or is formed in the alveolar shape is omitted or remarkably reduced, the neck of the cerebral aneurysm may be precisely ligated, thus allowing a surgical procedure to be more safely performed.

Further, the invention is innovative in that the tip of the cerebral aneurysm clip does not damage other blood vessels, or does not ligate other blood vessels together during the process of ligating the cerebral aneurysm. Therefore, if the ligation part of the cerebral aneurysm clip is made in the shape of the closed end to reduce the possibility of the above-mentioned accidents, it is unnecessary to dangerously pull the cerebral aneurysm so as to check the backside of the cerebral aneurysm, and the effort and risk of additionally using a mirror and a contrast medium can be reduced.

In addition, even a giant aneurysm from which the blood vessel that is the cerebral-aneurysm origin, which is difficult to find a dissection portion, an aneurysm origin or the like, is invisible can be treated with ease.

Further, since the ligation part of the cerebral aneurysm clip according to the present invention is made in the shape of the closed end to apply a force to the ligation part and evenly maintain the force, the ligation part is not prevented from being loosened when clipping a hard cerebral aneurysm such as the arteriosclerosis, and consequently stability can be further enhanced.

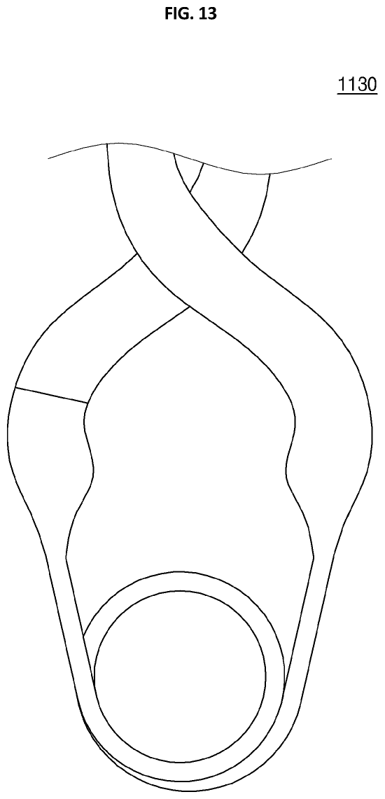

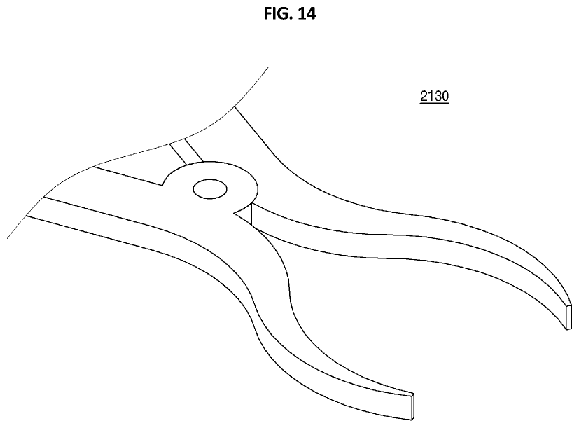

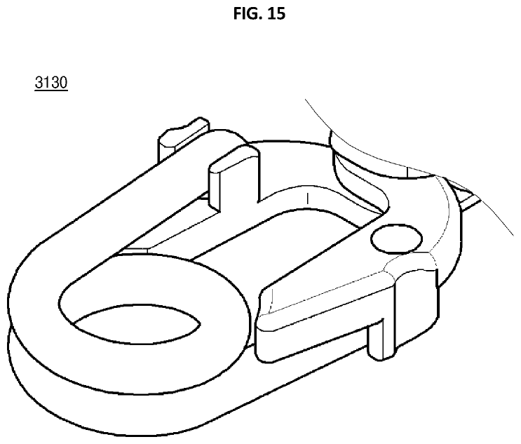

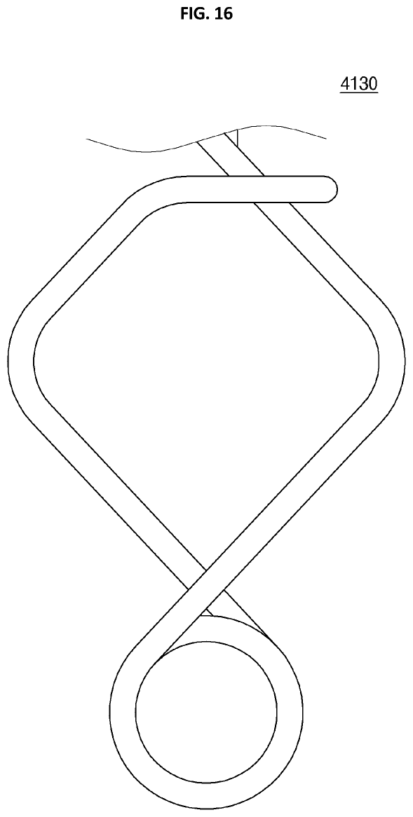

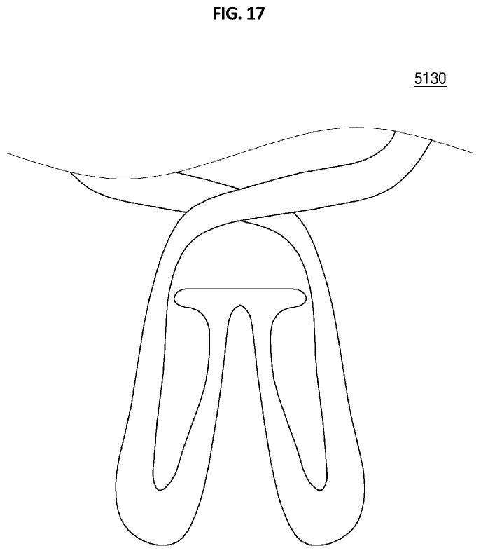

FIGS. 13 to 17 are diagrams illustrating various embodiments of the tong part 130 according to the present invention. The tong part 1130, 2130, 3130, 4130, or 5130 may be variously formed. If a force is applied to the external portion of the tong part 1130, 2130, 3130, 4130, or 5130, a distance between the first ligation part 110 and the second ligation part 120 is increased to define a space in which the cerebral aneurysm is ligated. As such, the tong part 130 may be changed in various ways, and the scope of the invention is not limited to the listed embodiment. It is obvious that other shapes of tong parts fall within the purview of the invention.

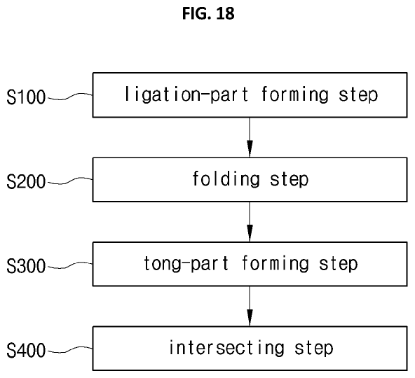

FIGS. 18 to 20 show methods of manufacturing cerebral aneurysm clips according to the present invention. Hereinafter, the methods of manufacturing the cerebral aneurysm clips according to the present invention will be described.

Referring to FIG. 18, the manufacturing method of the cerebral aneurysm clip according to the present invention includes a ligation-part forming step s100, a folding step s200, a tong-part forming step s300, and an intersecting step s400.

Hereinafter, the first and second embodiments of the cerebral-aneurysm clip manufacturing method will be described.

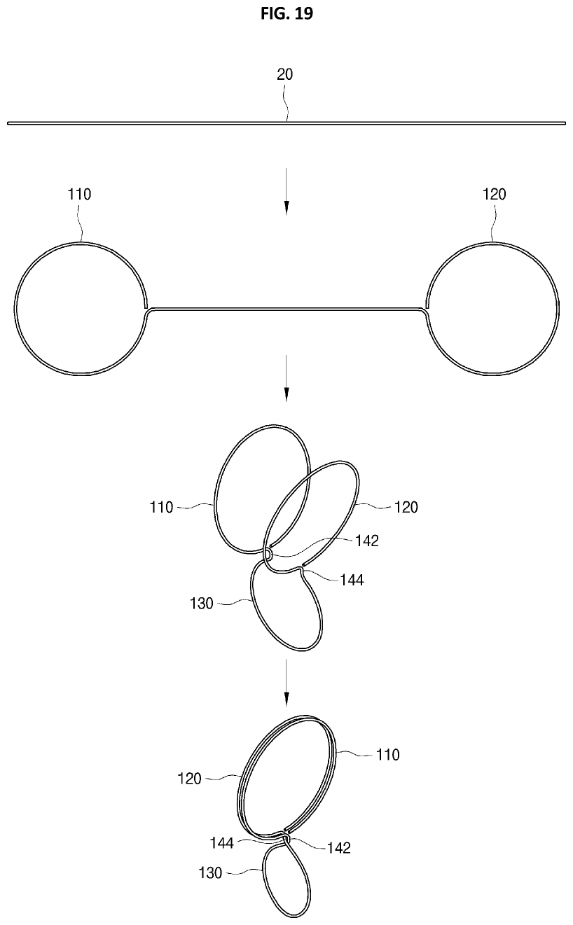

FIG. 19 sequentially shows the cerebral-aneurysm clip manufacturing method according to the first embodiment. According to the first embodiment, a straight line-shaped member having predetermined elasticity as the material of the clip is used for the base part 20.

At a ligation-part forming step s100, a first ligation part 110 and a second ligation part 120 having a symmetrical shape are formed on both ends of the base part 20. The first ligation part 110 and the second ligation part 120 are provided to form the closed end shape. By way of example, the first ligation part 110 and the second ligation part 120 may have a circular or elliptical shape. Further, they have various closed end shapes such as a rectangular shape or a diamond shape. The first ligation part 110 and the second ligation part 120 may be symmetrical with respect to each other while facing each other.

At a folding step s200, the base part 20 is bent such that the first ligation part 110 and the second ligation part 120 face each other. The first ligation part 110 and the second ligation part 120 are spaced apart from each other by a predetermined distance to define a space therebetween. A blood vessel that is to be ligated is located in this space.

At a tong-part forming step s300, a tong part 130 is formed between the first ligation part 110 and the second ligation part 120. The tong part 130 may be formed to have a circular or elliptical shape. A plane of the tong part 130 may be formed to be perpendicular to planes of the first ligation part 110 and the second ligation part 120.

Here, the tong part 130 and a connection part 140 may be formed together. The connection part 140 may be formed on both ends of the tong part 130. A first connection part 142 may be formed to connect the tong part 130 with the first ligation part 110, while a second connection part 144 may be formed to connect the tong part 130 with the second ligation part 120. The first connection part 142 and the second connection part 144 each may have a horizontal straight-line shape.

At the intersecting step s400, the first connection part 142 and the second connection part 144 may intersect with each other. While the facing first ligation part 110 and second ligation part 120 intersect with each other, one of the first ligation part 110 and the second ligation part 120, which is located relatively forwards, may be positioned behind the other. By way of example, if the rear surface of the first ligation part 110 faces the front surface of the second ligation part 120 when the tong-part forming step s300 has been completed, the front surface of the first ligation part 110 may be arranged to face the rear surface of the second ligation part 120. Hence, the first ligation part 110 and the second ligation part 120 may have the function of the tongs. In other words, if the external portion of the tong part 130 is pressed, the first ligation part 110 may move away from the second ligation part 120. Further, since the base part 20 has the elasticity, if the force applied to the tong part 130 is eliminated, the distance between the first ligation part 110 and the second ligation part 120 is reduced, thus ligating the cerebral aneurysm.

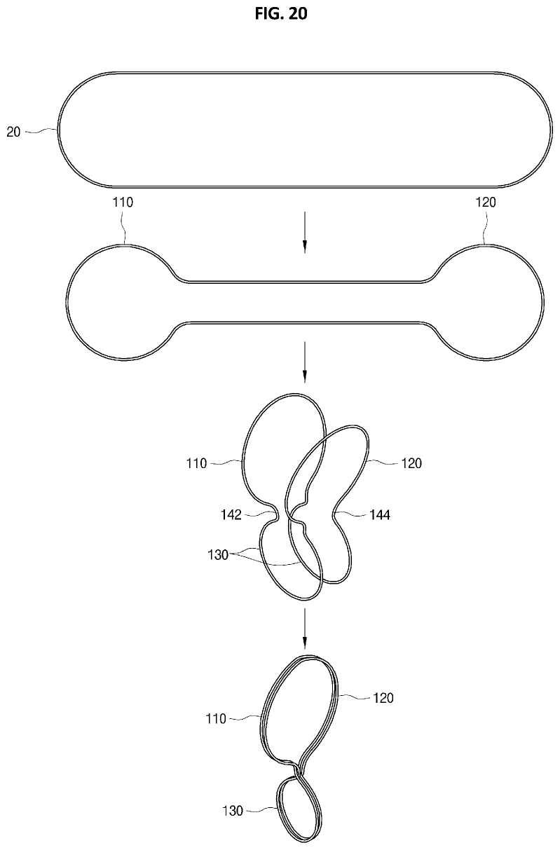

FIG. 20 sequentially shows the cerebral-aneurysm clip manufacturing method according to the second embodiment. According to the second embodiment, an elliptical closed end-type member having predetermined elasticity as the material of the clip is used for the base part 20. Since the subsequent steps, namely, the folding step s200, the tong-part forming step s300, and the intersecting step s400 remain the same as those of the above-described first embodiment, a detailed description thereof will be omitted herein.

However, the tong part 130 has a double structure, unlike the above-described first embodiment. At the intersecting step s400, the first connection part 142 and the second connection part 144 intersect with each other such that the front surface of the first ligation part 110 and the rear surface of the second ligation part 120 face each other. Hence, if the external portion of the tong part 130 is pressed, the first ligation part 110 may move away from the second ligation part 120. Further, since the base part 20 has the elasticity, if the force applied to the tong part 130 is eliminated, the distance between the first ligation part 110 and the second ligation part 120 is reduced, thus ligating the cerebral aneurysm.

While the present invention has been particularly described with reference to exemplary embodiments shown in the drawings, it will be understood by those of ordinary skill in the art that the exemplary embodiments have been described for illustrative purposes, and various changes and modifications may be made without departing from the spirit and scope of the present invention as defined by the appended claims.

### DESCRIPTION OF REFERENCE NUMERALS

| | |
|---|---|
| 100: cerebral aneurysm clip | 110: first ligation part |
| 120: second ligation part | 130: tong part |
| 140: connection part | 150: elastic member |

1. A cerebral aneurysm clip for ligating a cerebral aneurysm, the cerebral aneurysm clip comprising:
a first ligation part having a first hole formed therein, and having a closed end shape in which an edge of the first hole is closed;
a second ligation part having a second hole formed therein, and having a closed end shape in which an edge of the second hole is closed; and
a tong part connected with the first ligation part and the second ligation part, and applying a force such that the first ligation part and the second ligation part may be ligated on the cerebral aneurysm,
wherein the first ligation part and the second ligation part are arranged to face each other, such that the first ligation part and the second ligation part are ligated on both sides, respectively, with the cerebral aneurysm therebetween, and
the first hole and the second hole communicate with each other, and the cerebral aneurysm is positioned and ligated in the first hole and the second hole.

2. The cerebral aneurysm clip of claim 1, wherein the tong part has predetermined elasticity, and
a distance between the first ligation part and the second ligation part is increased if a force is applied to an external portion of the tong part, and the distance between the first ligation part and the second ligation part is reduced if the applied force is removed from the external portion of the tong part.

3. The cerebral aneurysm clip of claim 2, further comprising:
a first connection part connecting the first ligation part with the tong part; and
a second connection part connecting the second ligation part with the tong part,
wherein the first connection part and the second connection part intersect with each other.

4. The cerebral aneurysm clip of claim 3, further comprising:
an elastic member coupled at both ends thereof to the first connection part and the second connection part,
wherein the elastic member provides an elastic force in a direction for reducing the distance between the first ligation part and the second ligation part, when the cerebral aneurysm is ligated on the first ligation part and the second ligation part.

5. A method of manufacturing a clip for ligating a cerebral aneurysm, the method comprising:
forming a first ligation part and a second ligation part, each having a closed end shape, on both sides of a base part having predetermined elasticity to be symmetrical with respect to each other;
folding the base part such that the first ligation part and the second ligation part face each other;
forming a tong part between the first ligation part and the second ligation part, and forming a first connection part connecting the first ligation part with the tong part, and a second connection part connecting the second ligation part with the tong part; and
intersecting the first connection part and the second connection part with each other such that a front surface of the first ligation part and a rear surface of the second ligation part face each other.

6. The method of claim 5, wherein the base part is a straight line-shaped member having predetermined elasticity.
